(19) United States
(12) Patent Application Publication
(10) Pub. No.: US 2010/0216230 A1
(43) Pub. Date: Aug. 26, 2010

(54) Systems for Increased Cooling and Thawing Rates of Protein Solutions and Cells for Optimized Cryopreservation and Recovery

(75) Inventors: Robert E. Thorne, Ithaca, NY (US); Scott McFarlane, Manlius, NY (US); Matthew Warkentin, Ithaca, NY (US)

Correspondence Address: JONES, TULLAR & COOPER, P.C., P.O. BOX 2266 EADS STATION, ARLINGTON, VA, 22202, US

(73) Assignee: CORNELL RESEARCH FOUNDATION, INC., Ithaca, NY (US)

(21) Appl. No.: 12/443,199

(22) PCT Filed: Sep. 28, 2007
(86) PCT No.: PCT/US2007/079996

§ 371 Date: May 5, 2010

Related U.S. Application Data
(60) Provisional application No. 60/847,666, filed on Sep. 28, 2006.

(51) Int. Cl. C12M 1/04; F25D 31/00; F17C 7/02

(52) U.S. Cl. 435/307.1; 62/293; 62/52.1

(57) ABSTRACT

In systems and methods for freezing and subsequently thawing liquid samples containing biological components, a sample is fractioned into a very large number of small drops (10) having surface area to volume ratios of 1000 m-1 or greater. The drops are projected at a liquid cryogen (40) or at the solid surface of a highly thermally conducting metal cup or plate, where they rapidly freeze. The cold gas layer that develops above any cold surface is replaced with a dry gas stream (75). The environmental temperature experienced by the sample then abruptly changes from the warm ambient to the temperature of the cryogenic liquid or solid surface. To thaw drops with the highest warming rates, the frozen drops may be projected into warm liquids. The sample is projected with cold gas to the warm liquid, so that again there is an abrupt transition in the environmental temperature.

### CROSS REFERENCE TO RELATED APPLICATIONS

This application claims the benefit, under 35 U.S.C. 119(e), of U.S. Provisional Application No. 60/847,666, filed Sep. 28, 2006, which is hereby incorporated by reference in its entirety.

### BACKGROUND OF THE INVENTION

1. Field of the Invention

The present invention relates in general to apparatus and methods for rapidly freezing and thawing proteins, cells and other biological molecules for optimizing the cryopreservation thereof.

2. Description of the Background Art

Cryopreservation of proteins and other biological molecules, of cells and of tissues plays an important role in modern biology and medicine. However, the cryopreservation process itself may damage or degrade the samples, so that there is a strong incentive to develop improved methods and hardware.

Cryopreservation of Protein Crystals

In the case of protein crystals, which are very fragile structures held together by non-bonding weak intermolecular interactions, we have shown that there are two important factors controlling the amount of damage caused by cooling: (1) the cooling rate, and (2) the extent to which crystal components have similar thermal expansion/contraction behavior. Faster cooling produces less damage, and we have conjectured (but not explicitly shown) that matching thermal expansion behavior of the crystal components can reduce damage.

Another major issue in cryopreservation of protein crystals is that smaller crystals (less than 100 micrometers) rapidly dehydrate in ambient air because of their very large surface area to volume ratio. The amount of dehydration in the 1-3 seconds between mounting and plunging in the liquid cryogen can be sufficient to significantly alter the solvent content, structural properties and X-ray diffraction properties of 10-50 micrometer crystals. Juers and Matthews have shown that condensation and freezing of water vapor from ambient air onto cold crystals can lead to significant changes in solvent content when the crystals are thawed.

Cryopreservation of Protein Solutions

Cryopreservation of proteins can in principle allow experiments using protein from a single source to be reproduced or extended months or years after initial protein production.

Long-term storage of proteins is a significant issue in structural genomics and protein crystallography. Solutions of some robust proteins such lysozyme can be successfully flash frozen and dried to yield the lyophilized products sold by, e.g., Sigma Chemical. But many proteins and protein complexes, including those of greatest scientific interest, cannot survive this process without loss of structural and/or functional integrity. Proteins stored above 273 K are subject to oxidation, proteolysis, and aggregation. Purified protein solutions with large added cryoprotectant concentrations can be bulk frozen, but the cryoprotectants must be removed after thawing.

An alternative procedure is to drop 20-50 microliter volumes of protein directly into liquid nitrogen, and then remove the frozen pellets for storage. Unfortunately, following a freeze-thaw cycle many if not most protein solutions show significant aggregation and precipitation, and their crystallization behavior (which is strongly affected by the presence of aggregates and other “impurities”) may be completely different. For this reason, most protein crystallographers strongly prefer to use fresh protein in crystallization trials, and to grow up fresh protein for each experiment. The costs, in terms of media, time, and the inability to run duplicate experiments at later dates, are enormous. A single preparation that yields a few milliliters of, e.g., a membrane protein can cost $50,000.

Freezing and thawing protein solutions often degrade protein function as measured, for example, by assays of enzymatic activity. This is a significant problem in biochemical studies, and has consequences for the long-term storage of protein-based drugs.

The problems of cryopreserving proteins arise from several sources. All physico-chemical properties of the protein, solvent and other solution components—including pH, solubility, the activity and viscosity of water—vary with temperature. Cooling at modest rates allows relaxations and redistributions that lead to an inhomogeneous low temperature state, with regions that are solvent rich and solvent poor, salt rich and salt poor, protein rich and protein poor. These inhomogeneities promote protein aggregation and denaturation.

Frozen samples are typically stored at 193 K, well above water's glass transition. Significant solvent and solute diffusion can occur on storage time scales of weeks that enhance sample inhomogeneities. Cooling in liquid nitrogen likely produces vitreous ice, but at these high storage temperatures it readily transforms to cubic ice.

These problems are compounded on thawing. Heat transfer in standard methods is less efficient than during cooling and the time required to thaw is much longer. Even if the solution was initially cooled into the glass phase, it transforms to cubic and then hexagonal ice before finally melting. Since crystalline ice incorporates very different concentrations of solutes like salts and protein than the background “solution” from which it grows, additional sample inhomogeneities result. These microscopic inhomogeneities (such as salt and/or protein-rich pockets) together with the relatively slow warming towards room temperature can then drive protein out of solution and/or destabilize its conformation, leading to aggregation and precipitation.

In current best practice, protein solution is frozen in drops or in PGR tubes in 20-50 microliter volumes. Cooling times are on the order of seconds, and thawing times, although not reported, are certainly longer. The results obtained using these and other methods are severely deficient. A method that allowed large volumes of protein solution—for example, the entire volume produced in a single expression run—to be successfully cryopreserved would have a major impact on many areas of biotechnology and biological research.

Cryopreservation of Cells

Cryopreservation of sperm and egg cells is essential for propagation of animals by artificial insemination, in human fertility treatments, and in preservation of endangered species. A wide variety of other cell types including stem cells are routinely cryopreserved. However, current methods for cryopreserving all of these systems are severely deficient, in that survival rates of cells and of important cell functions are highly variable and often extremely poor. The issues are largely similar to those in cryopreservation of proteins, with the added complication that stresses due to differential expansion of cell components, growth of ice crystals inside and outside the cell, and osmotic pressure gradients across cell membranes can rupture membranes and other cellular structures, causing loss of function.

The methods used to cryopreserve sperm are typical. Ejaculate is collected and evaluated for sperm count and motility. The ejaculate is then centrifuged, a pellet of sperm cells collected, and then extenders including glycerol (for cryoprotection), egg yolk, and food detergents added to the pellet. The sperm mixture is then dispensed into straws with volumes typically between 0.5 ml (for humans) to 5 ml (for large animals like horses.) Straws are then placed on a freezing rack set above liquid nitrogen, and after 20 minutes are then placed directly in liquid nitrogen. The frozen straws are then transferred to a liquid nitrogen tank for storage. Programmable coolers are commercially available to automate the process. The sperm mixture is then thawed by placing the straws in warm water, with typical thaw times of several seconds. Survival rate depends strongly on thaw time and temperature.

A major problem with these current methods is that the cooling rate of the sperm mixture is extremely slow (5-50 K/minute)—requiring tens of seconds to minutes to cool below water's glass transition temperature of 150 K. To cool cells so slowly and still avoid hexagonal or cubic ice formation inside or outside, very large concentrations of cryoprotectants—which are much more likely to have deleterious effects on the cells—must be used. This slow cooling also allows migration of solutes and solvent into and out of the cells as chemical potentials for various species evolve as the temperature drops. On thawing, this latter process is reversed. Moreover, the slow (5-10 s) thawing may allow vitreous ice to transform to cubic or hexagonal ice before finally melting, causing cell damage. Detailed models of the cryopreservation process have been developed, but often rely on equilibrium ideas that are not appropriate when cooling is fast.

Single and few sperm freezing have been performed using sperm injected into empty eggs and traditional cooling methods, and very recently by direct immersion of sperm collected on a Cryoloop (used to hold protein crystals) into liquid nitrogen. For this latter application. MicroMounts developed by our group (and now commercially available from Mitegen. LLC) are likely to be far superior to Cryoloops in both ease of handling and performance.

At present there is no reliable way to rapidly cool large quantities of sperm (or other cells), such as may be contained in the volume of a single ejaculation.

Routes to Faster Cooling

A number of methods have been proposed to increase the cooling rates of liquid samples, including decreasing the sample volume, increasing the speed with which the sample is directed at a cold liquid or solid, and using slushed liquids or metals as the cooling medium.

In general, small sample volumes are expected to cool more rapidly than large volumes. Samples are thus commonly atomized or nebulized into a spray of fine drops. Atomizers and nebulizers generally provide poor control over drop volume, and give a wide distribution of volumes within the spray. Since cooling rate varies with drop volume, the drops within a given sample may exhibit a wide range of freezing behaviors.

Drop sizes from typical atomizers and nebulizers can be 10 to 250 micrometers, corresponding to volumes of picoliters to nanoliters. If these drops are sprayed in a dry atmosphere, significant evaporation from each drop can occur during the transit from nozzle to cold surface, producing significant deviations in protein and other solute concentrations from those in the original solution. Similarly, if the drops are sprayed in a humid atmosphere, they may take up excess water from the atmosphere, and water vapor will freeze out on the cold surface with the drops. When the sample is thawed, this water will mix with the sample, diluting it. This dehydration and condensation make the actual concentrations in the frozen and thawed drops unknown, and thus make it very difficult to design reliable cryopreservation and recovery protocols.

Atomizers and related devices that blow air through a liquid to produce a fine spray of drops lead to drops with higher dissolved oxygen concentrations than the original solution, which can have deleterious effects on the thawed sample. Oxygen promotes faster oxidation of biological molecules and cells and faster sample degradation.

Projecting liquid samples from nozzles at high speed can introduce significant shear forces, which are known to damage cells. Additional stresses can occur when drops are frozen on cold metal or other solid surfaces. Heat transfer to the solid can be so efficient that large thermal gradients may develop across the sample itself as it cools. Heat transfer from drops cooled in liquid cryogens tends to be limited by the fluid boundary layer, and so internal temperature gradients tend to be smaller.

The choice of cooling medium is also important. The sample can be cooled in a cold gas stream (e.g., N₂ at T=100 K). Cooling rates can be increased by decreasing the gas temperature or increasing the gas velocity. The sample can be plunged into a liquid cryogen such as liquid nitrogen, propane or ethane, and cooling rate may increase with plunge speed. Another known technique uses slushed liquids, held at their melting temperature, to take advantage of extra cooling provided by the latent heat of fusion. In practice, the extra cooling provided by slushes is only relevant for very large samples; for the small volume drops of interest in, e.g., biotechnology, the increase in cooling rate over that provided by the liquid is negligible because heat transfer is limited by the thin layer of liquid coating the solid particles in the slush.

Fast cooling can be achieved by projecting (“splatting”) the sample onto a cold solid surface such as solid copper, whose high thermal conductivity results in excellent heat transfer from the surface to the drop in contact with the metal.

Compared with the attention focused on fast cooling, fast thawing has received very little attention. This is surprising, since most of the same processes leading to sample degradation during cooling are also operative during warming. In current practice, the sample and its container (e.g., a centrifuge tube) are typically immersed in a warm liquid. This is true even when the sample has been frozen as pellets.

### SUMMARY OF THE INVENTION

The present invention comprises systems and methods for freezing and subsequently thawing liquid samples containing biological components such as proteins and cells. These systems and methods yield much larger cooling and thawing rates for a given drop volume, more reproducible and controllable cooling and thawing rates, reduced evaporation/dehydration and oxygen contamination, and reduced shear forces. They allow faster cooling and thawing with larger drops and smaller drop velocities. The cooling and thawing processes experienced by each drop are much more reproducible, and the initial drop solute concentrations are preserved throughout the cooling and thawing process.

The sample is fractioned into a very large number of small uniform separated drops of volume between 0.01 nl and 10 ul having surface area to volume ratios of 1000 m⁻¹ or greater using conventional liquid handling/drop dispensing devices or flow cytometer technology rather than atomizers or nebulizers. These produce drops of reproducible volumes down to ˜100 nanoliters and ˜100 picoliters, respectively. Unlike atomizers and nebulizers, they do not entrain the drops in gas and so do not increase the dissolved gas—and specifically oxygen—content. In the preferred embodiments, these drops are projected at a liquid cryogen or at the solid surface of a highly thermally conducting metal cup or plate, where they rapidly freeze. Frozen drops are stored in a suitable cryogen or in a cryogenic temperature container at low temperature, preferably below water's glass transition temperature of ˜150 K. To thaw drops with the highest warming rates, the frozen drops may be projected into warm liquids, for example a buffer solution which is friendly to the sample or a warm oil in which it is immiscible. Alternatively, drops frozen in very thin metal cups can be thawed by driving the cup onto a warm metal surface or into a warm liquid.

A crucial feature of the cooling method is the removal of the cold gas layer that develops above any cold surface and its replacement with warm, dry gas. The environmental temperature experienced by the sample then abruptly changes from the warm ambient to the temperature of the cryogenic liquid or solid surface. Similarly, on thawing, the sample is projected with cold gas to the warm liquid or solid surface, so that again there is an abrupt transition in the environmental temperature. These abrupt transitions ensure that all cooling and warming occurs in the medium that provides the greatest heat transfer rates and thus yields the fastest possible cooling and warming rates and the most reproducible time-temperature profiles. They allow even relatively small drop velocities relative to the cold surface to give fast cooling, reducing stresses on cells within the liquid.

The presence of the warm dry gas above the cryogenic liquid or solid allows the dispensing tip to be placed close to the cold surface, minimizing evaporation and dehydration of the drop during its flight to the cold surface. This eliminates the need to maintain a humid atmosphere, and thus eliminates water vapor condensation on the cold surfaces and dilution of the sample on thawing. Larger cooling rates can be obtained with larger drop volumes and smaller drop velocities. The environment within the cooling and thawing chambers can thus be filled with a warm dry gas like nitrogen.

### BRIEF DESCRIPTION OF THE DRAWINGS

The features and advantages of the present invention will become apparent from the following detailed description of a number of preferred embodiments thereof, taken in conjunction with the accompanying drawings, which are briefly described as follows.

FIG. 1 shows a side view of one embodiment of the present invention for cryopreservation of liquids containing biological materials, in which liquid drops are frozen in a liquid cryogen and cold gas above the liquid is removed using a warm dry gas stream.

FIG. 2 shows a side view of a second embodiment of the present invention in which drops are frozen onto the surface of a very thin, highly thermally conducting cup cooled by a liquid cryogen. The cold gas that forms above the cold surface is removed using a warm dry gas stream.

FIG. 3 shows another embodiment based upon the embodiment of FIG. 1, in which the freezing apparatus is contained in a chamber in which an atmosphere of warm dry gas is maintained.

FIG. 4 shows an embodiment of the thawing component of the present invention, compatible with the freezing embodiments in FIGS. 1 and 3. Frozen pellets are projected in a cold gas stream into a warm liquid.

FIG. 5 shows an embodiment of the thawing component of the present invention, compatible with the freezing embodiment in FIG. 2. The thin metal cup containing the frozen sample is projected in a cold gas atmosphere onto a warm metal surface, to which it is drawn by vacuum suction.

### DETAILED DESCRIPTION OF THE INVENTION

The systems and methods described here have considerable potential to improve the cryopreservation of protein solutions, cells and other biological samples. The precision and reproducibility of the cooling and thawing steps can be greatly improved, allowing greater control and easier optimization of cooling and thawing conditions for each sample. Maximum cooling and thawing rates for a given drop volume can also be dramatically improved, while at the same time minimizing dehydration, oxygen contamination and shear forces that may damage cells and degrade proteins.

Since cryopreservation involves both the freezing and subsequent thawing of a sample for later use, cryopreservation systems must necessarily involve both freezing and thawing components. In the present invention, a crucial insight that enables large improvements in both freezing and thawing performance with small drops is the use of methods to control the temperature in gas layers above cold and warm surfaces.

As amply reflected in the prior art, fractioning a sample into small drops is expected to increase the cooling rates, and smaller drops are expected to give faster cooling rates. We have shown that in typical experimental set-ups this is not true, and that below drop volumes of roughly 1 microliter, cooling rates become nearly independent of drop volume. The cause of this saturation is the cold gas layer that forms above any cold surface (as described in U.S. Application No. 60/787,206, filed Mar. 30, 2006, which is hereby incorporated by reference and is hereinafter referred to as the 206 application). Saturation of cooling rates at even larger volumes may occur when the walls of the cooling apparatus are well insulated and produce thicker gas layers

Freezing by Cold Gas Layers

Cooling by thermal conduction, convection and radiation produces a layer of cold gas above any cold liquid or solid surface. Boiling of, e.g., liquid nitrogen at T=77 K adds to this cold gas layer. The thickness of the cold gas layer can be defined as the height above the liquid surface at which the temperature rises to, e.g., waters glass transition temperature (T₉˜150 K) or its homogeneous ice nucleation temperature (Tₕ˜231 K). The height of the gas layer depends upon the height of the confining walls above the cold liquid or solid surface. If there are no walls, the gas layer above liquid nitrogen may be 1 cm thick, but with well-insulated walls (as in liquid nitrogen storage dewars) the gas layer can extend 10-30 cm or more above the cold surface.

Large samples can pass through these cold gas layers with little change in their internal temperatures, so that nearly all cooling occurs once the sample enters the cold liquid, with large heat transfer rates typical of cryogenic liquids and slow cooling rates because of the large sample mass). But for sufficiently small drops—less than about 1 microliter or a diameter of roughly 1 mm for a 2 cm thick gas layer—most cooling of the drop will occur in the gas layer, not the liquid. The drop temperature will mirror the gas temperature as it passes through the gas, and the cooling rate will be determined by the much smaller heat transfer rate to the gas. As a result, measured cooling rates for small drops plunged into liquid nitrogen or liquid propane are one to three orders of magnitude smaller than one would expect based upon the drop size and the heat transfer properties of liquid cryogens.

The thickness of the cold gas layer can be influenced by factors such as environmental air currents and by the time between filling of a container with liquid cryogen and the dispensing of drops. They thus introduce a variable factor in cooling that affects reproducibility of experiments.

By removing this cold gas layer and replacing it with warm gas, where “warm” typically means warmer than the sample's glass transition temperature, its homogeneous ice nucleation temperature, or its melting temperature, an abrupt transition from warm gas to cold liquid can be created along the sample's path. Consequently, the cooling rate will be independent of the time to traverse the gas above the cold liquid or solid surface, and the sample will begin cooling only once it enters the cold liquid or reaches the cold solid surface, at the high cooling rate provided by the liquid cryogen By eliminating the cold gas layer, cooling rates for small drops can be increased by one to three orders of magnitude, from ˜100 K/s to 10.000 K/s or 100,000 K/s, depending upon drop volume, liquid cryogen and plunge speed through the liquid.

Thawing by Warm Gas Layers

The same phenomena are relevant during thawing of small drops. When frozen drops are projected into warm liquids or onto warm solid surfaces, the warm gas layer above the surface can cause small drops to thaw in the gas, at rates determined by the relatively slow heat transfer rates to the gas. Replacing this warm gas with gas at the initial temperature of the frozen drop again can produce an abrupt temperature transition at the gas-liquid or gas-solid interface, and ensure that thawing occurs only once the drop enters the warm liquid or hits the solid surface, with the much larger heat transfer rates of the liquid or solid.

The present invention comprises methods and apparatus designed to allow very large cooling and thawing rates (100-100,000 K/s) of proteins, cells and tissues. The keys to achieving these objectives are:

(1) Maximizing heat transfer rates from the sample by maximizing its surface to volume ratio and using cold gas layer removal. The cooling/warming rate increases rapidly with increasing surface to volume ratio. From experiments performed in our laboratory, cooling rates in the 100 K/s to 100,000 K/s are likely to give the best outcomes, requiring the smallest cryoprotectant concentrations and giving most homogeneous samples. This requires that the sample be fractioned into volumes of 1 microliter or smaller (corresponding to sample surface-to-volume ratios of ˜1000 m⁻¹ or larger) with 1 microliter given cooling rates of ˜100-1000 K/s and 0.01 nanoliters (approaching the size of individual cells) giving cooling rates approaching 100,000 K/s.

(2) Maximizing heat transfer rate by proper choice of cooling method.

(3) Obtaining highly controllable cooling and warming.

(4) Cooling a large total sample volume (e.g., ml or larger) in a modest time compared with the time required for sample degradation at ambient temperature.

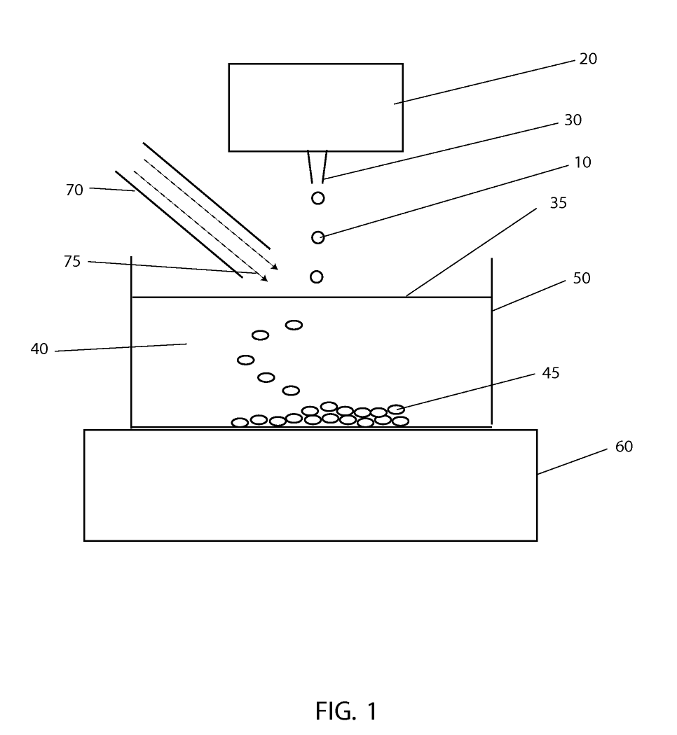

FIG. 1 shows one preferred implementation of the freezing part of this cryopreservation system. The liquid sample to be preserved may consist of water, salts, sugars, buffers, alcohols, cryoprotectants like glycerol, proteins and cells, among other components. Since cooling and warming rates in the present method are dramatically increased over most prior methods, the concentrations of cryoprotectants needed to prevent hexagonal ice formation is reduced.

The protein or cell mixture is then dispensed in microliter or submicroliter (between 0.01 nl and 10 ul) drops 10 of similar size and with a surface area-to-volume ratio of ˜1000 m⁻¹ or larger, until the entire sample volume has been frozen. Drops of similar size will undergo similar freezing and thawing, and thus provide well defined conditions for cryopreservation. A non-contact liquid dispenser 20 with a single tip 30 or multiple tips can be a manual pipeter. It can be an automated liquid handling/drop dispensing device (based upon, for example, mechanical displacement (e.g., syringe pumps), thermal heating, hydrostatic pressure jumps) such as are used in protein crystallization. This technology can dispense hundreds of drops per minute per channel/tip, and can be equipped with multiple tips for parallel dispensing. Drops with volumes down to about 100 nanoliters can be dispensed with little fractional volume error and drops as small as 10 nanoliters can be dispensed using current technology. An example of commercial dispensing technology that can be adapted for this purpose is the Honeybee 161/181 from Genomic Solutions of Ann Arbor, Mich., (which uses precision syringe pumps to dispense 0.05 to 1 microliter drops). Other manufacturers of suitable technology include Perkin Elmer, Art Robbins Instruments, Beckman-Coulter, and Innovadyne.

Continuous streams of smaller drops—with less control over drop spacing—can be produced using flow cytometer technology. Typical flow cytometer dispensing tip apertures range from 50 to 700 micrometers, corresponding to drop sizes of 0.1 nanoliters to ˜300 nanoliters. A variety of microfluidic devices have been demonstrated to produce even smaller drops. For 100 nanoliter drops, a 1 ml sample will require dispensing 10,000 drops; for 1 nanoliter drops, a 1 ml sample will require 1,000,000 drops. These can be dispensed in a few minutes or less using commercial liquid handling technology. The technology provides excellent volume accuracy, ensuring uniform and reproducible drop volumes and cooling and thawing outcomes. Some cells are thought to be sensitive to shear forces, which can be reduced by reducing the dispensing velocity and/or increasing the dispensing tip diameter.

The liquid drops 10 then travel from the tip 30 to a horizontal top surface 35 of a cold liquid 40, and then on into the bulk of the liquid where they are frozen. Because the drops are denser than liquid cryogens, they will settle to the bottom of the container as hard pellets 45. These pellets can then be collected and stored at cryogenic temperature (preferably well below T_{g}=150 K) for subsequent thawing.

The cold liquid 40 may be any liquid cryogen including nitrogen, propane, and ethane or any liquid refrigerant. Liquid nitrogen is adequate for small (<0.1 microliter) drops. For larger drops, liquid propane or ethane can provide faster cooling.

The cold liquid 40 is contained in an insulated container 50, which then also serves as a container for the frozen drops. This container can be rotated or translated by a stage 60, to ensure that successive drops freeze independently in different parts of the liquid and do not agglomerate, ensuring the fastest cooling rates.

A nozzle or tube 70 projects a stream of warm, dry gas 75 along the surface 35 of the liquid cryogen so as to remove the cold gas layer that forms above it. Details are described in the previously mentioned '206 patent. The gas should be free of any constituents having boiling or melting temperatures above that of the liquid cryogen, to prevent condensation and build-up of these constituents in the liquid cryogen and contamination of the drops. When liquid nitrogen is used as the cooling medium, dry nitrogen gas is good choice, and has the advantage of eliminating the possibility of oxygen contamination that may promote degradation of the biological constituents on warming.

“Warm” here means warm compared with the temperature of the liquid cryogen. Suitable temperatures include the initial temperature of the liquid to be frozen, a few degrees above the melting temperature of that liquid, and a few degrees above the homogeneous nucleation temperature, depending on whether it is desired to precool the liquid before freezing to obtain the shortest cooling time through the most critical temperature region.

The character of the nozzle or gas outlet 70, the direction of the gas flow and the flow speed can vary considerably and still provide effective gas layer removal. The small area nozzle inclined at an angle to the surface area can direct a stream 75 of flowing gas to the region where drops are dispensed. Larger area outlets on either side of the container can produce a slow, nearly laminar and tangential flow across the liquid's surface. With larger area flows or with tips and nozzles placed close the liquid surface, the flow speed of the gas can be quite small so as not to appreciably disturb the trajectories of smaller drops.

Since the cold gas layer requires a finite time to reform, the warm gas flow need not be continuous, but instead can be pulsed on and off periodically, for example removing the cold gas just before drops are dispensed. The replacement of the cold gas layer with this warm gas helps prevent the dispensing tip from freezing, allowing it to be placed very close to the surface of the cold liquid. Minimizing the distance from dispensing tip to the cold liquid or solid surface will prevent evaporation and concentration changes in small drops during dispensing. In the preferred embodiments, the distance between the dispenser tip 30 and the cold surface 35 is between 1.0 and 10.0 cm.

By removing the cold gas layer, much larger cooling rates for a given drop size can be achieved. As a result, the minimum drop size can be increased, reducing the risks from evaporation and oxygen incorporation.

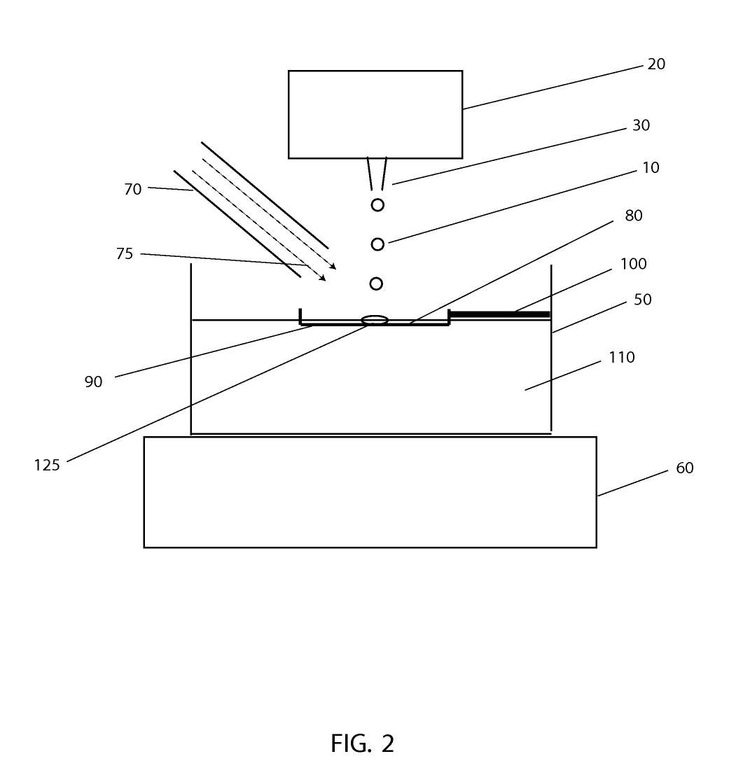

FIG. 2 shows an alternative embodiment in which the liquid cryogen is replaced by a cold solid horizontal surface 80, although it should be understood that the surface 80 could be concave as well. In a preferred embodiment, this solid surface is provided by a cup 90 formed from a very thin metal, held in place by an arm 100 with its bottom immersed in a bath of liquid cryogen 110. Alternatively, the cup 90 may be in contact with a large metal block cooled using a liquid cryogen or a closed cycle cryogenic refrigerator. The use of a thin cup (200 micrometers or less and preferably 25 micrometers) is useful on thawing, as it reduces the thermal mass and maximizes the heat transfer rate from the warm substrate to the sample. The cup 90 may have a variety of shapes, including a conical shape or hemispherical shape. The cup 90 together with the cold liquid or solid with which it is in contact may be rotated and translated using a stage 120 to ensure that successive drops fall on a fully cold surface and to minimize the overall thickness of the sample coating on the surface.

To achieve the fastest cooling, the thermal conductivity to the surface 80 should be maximized by using, e.g., a metal like copper. The cold surface 80 can be coated with an ultra-thin layer of, e.g., a Teflon-like (PTFE) polymer, or another, more inert metal like gold to prevent contamination and excessive adhesion of the frozen sample on warming.

Once the metal surface has been covered with frozen drops 125, heat transfer to drops that are subsequently dispensed will occur through the frozen layer, and will decrease as the layer thickness increases. This will set a practical limit on layer thickness, as will the requirements for warming rates during thawing.

Cooling rates in the liquid or on the solid can be increased somewhat by increasing the drop speed. Drops can be projected downward by applying an impulsive force (e.g., a pressure jump) inside the dispensing tip 30. Larger drop velocities may also reduce dehydration during transit from tip to cold surface and reduce cooling in any residual layer of cold gas. However, this may produce a large shear in the fluid as it is dispensed, which can be damaging to cells. At high speeds the impact of the drop with the cooling surface may yield irregularly shaped drops and introduce large transient shear forces that may be damaging to cells.

By removing the cold gas layer, all cooling before the sample reaches the cooling liquid or solid surface is eliminated. Consequently, the effect of drop speed on cooling rate is greatly diminished, and drop speeds can be reduced and still achieve larger cooling rates.

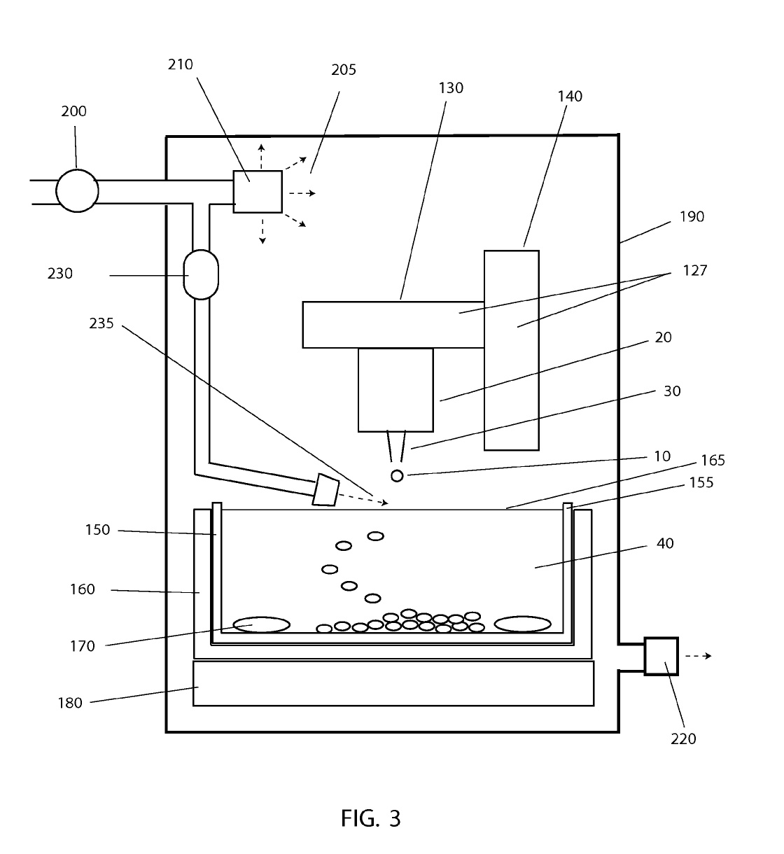

FIG. 3 shows an alternative preferred embodiment based upon the embodiment in FIG. 1. In this embodiment the dispenser 20 and dispensing tip 30 are mounted on a stage 127 with horizontal 130 and vertical 140 translation components. Side-to-side motions allow rastering of the tip 30 and thus the drops 10 across the surface of the cold liquid, ensuring that they freeze independently, are well separated, and do not agglomerate. Side-to-side drop deflection could also be achieved using a concentric ring of gas jets situated below and coaxially with the tip, using electrostatic deflection, or by pivoting the tip.

The vertical translation components 140 allow the height of the tip 30 above the surface of the cold liquid 40 to be adjusted. It also allows the tip and drop to be accelerated downward during dispensing. The initial drop velocity relative to the tip remains small but the drop velocity relative to the liquid is increased. This may give faster cooling without increasing shear forces during dispensing. With a tip velocity of 1 m/s (easily achievable using, e.g., a stepper motor, a linear motor, solenoid drive, piezo drive, pneumatic drive, etc.), the time for the drop to reach the liquid from a 10 cm height will be reduced to less than 0.1 s. Evaporation during dispensing will be negligible, but the impact of these drops will deform their shape.

The liquid cryogen and the frozen pellets in this case are held in a removable cup 150 that rests inside a thermally insulated container 160. The cold air that forms above the cold liquid will naturally flow around the rim 155 of the cup and down the sides of the container 160. Minimizing the height of the rim above the liquid surface 165 then minimizes the natural height of the cold gas layer, and minimizes the flow of warm dry gas required to eliminate it.

To maintain a uniform temperature in the liquid cryogen even when warm gas is blown across its surface, the liquid can be stirred or mixed. In FIG. 3, this is achieved using magnetic stir bars 170 placed in the bottom of the cryogen-containing cup, that are driven by a magnetic stirrer base 180. Other means of mixing include recirculating pumps and electric motor driven mixers.

To minimize condensation of moisture from the air onto cold surfaces, and in particular onto the liquid cryogen, where it may dilute the sample upon thawing, the apparatus may be enclosed in a chamber 190. A dry gas such as nitrogen enters the chamber through a pressure regulator 200. A continuous flow of dry gas 205 emanates from a diffuser 210 and exits the chamber through a one-way or release valve 220, carrying with it gas that has been cooled by contact with the cold liquid. A valve 230 controls the flow rate of dry warm gas 235 across the surface of the cold liquid. The use of an inert gas like nitrogen is useful for air or oxygen-sensitive samples.

To minimize storage requirements for each sample when liquid cryogens are used as the cooling medium as in the implementations of FIGS. 1 and 3, the frozen sample pellets can be transferred to a smaller container. This could be achieved by pouring the contents of the cup 150 through a sieve and then transferring the pellets to a smaller container that is then placed in a dry storage dewar. Pellets could also be automatically withdrawn and transported to another container using suction. To minimize storage requirements when drops are dispensed on solid surfaces as in the implementation of FIG. 2, a large number of very shallow cups can be sequentially moved into place for dispensing and then stacked for storage.

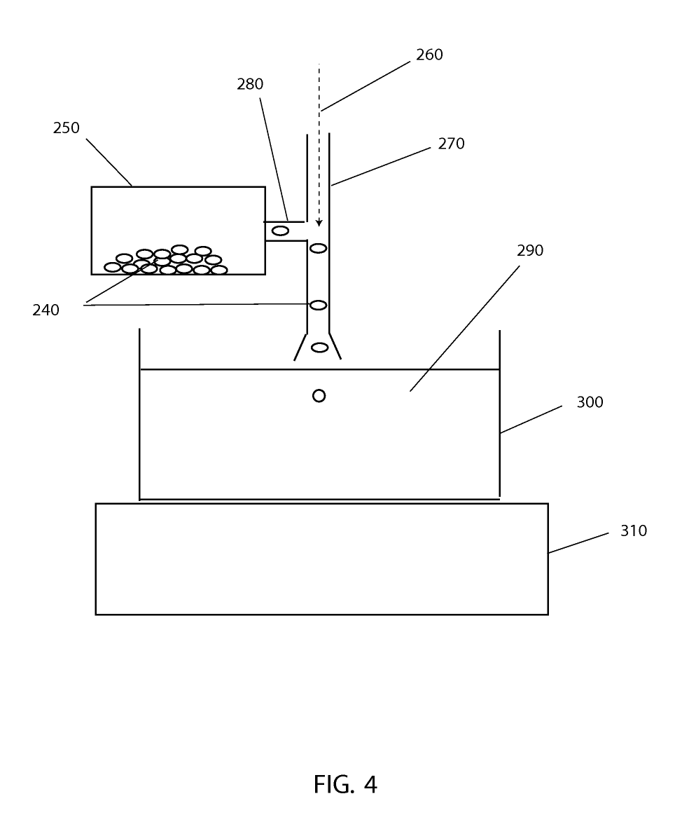

FIG. 4 shows how frozen drops/pellets produced by apparatus similar to those shown in FIGS. 1 and 3 may be rapidly thawed so as to capture the full benefit of rapid freezing and maximize the quality of the recovered sample. The frozen drops (pellets) 240 are stored in a cold insulated container 250, and transported from the container into a stream of cold flowing gas 260 contained in a tube 270. The temperature of the gas should be sufficient to maintain the temperature of the pellets below water's glass transition temperature T_{g} or its homogeneous ice nucleation temperature Tₕ throughout their trajectory to the thawing medium. The gas could be obtained from a pressurized container of liquid nitrogen. This flowing gas plays the same role as the flowing gas in the freezing apparatus, ensuring a uniform temperature along the pellets trajectory through the gas and an abrupt transition in temperature transition at the gas/thawing medium interface. All of the warming then occurs in the thawing medium and its high characteristic heat transfer rate. Both the gas flow speed and the pellet speed can be modest, since large speeds are not needed to prevent thawing on the way to the cooling medium.

The pellets may be transported from the container using suction created in another tube 280 by the cold gas flowing in tube 270, as in an atomizer, and this is facilitated by the small size of the pellets. They may be transported mechanically using an auger, using a gravity feed and vibration, or other methods commonly used in, e.g., the pharmaceutical industry to transport powders.

To rapidly thaw the pellets, they can be projected into a warm liquid 290 contained in a heated container 300. This liquid may be an aqueous buffer solution that is agreeable to the biological components of the pellets. The pellets will melt and release their biological components into the solution. The final solution then will have a smaller concentration of the biological components than the original solution that was frozen.

Alternatively, the pellets can be thawed in a warm liquid in which their constituents are immiscible, such as an oil or other hydrocarbon based liquid. In this case, the sample will melt into drops which will then density separate and coalesce, allowing the solution to be withdrawn at its initial, pre-freezing concentration.

To ensure the fastest warming, the liquid should be heated and mixed to compensate for cooling by the cold gas blowing on its surface and the pellets that melt within it. Mixing will also increase the relative motion of the pellets and liquid thawing and increase heat transfer rates. The mixer may be a magnetic stirrer, an electric motor driven blade, or a recirculating pump.

As with freezing, the container of warm liquid can be rotated or translated by the stage 310 as pellets are dispensed, to ensure that successive pellets thaw independently and thus with maximum heat transfer rates. Alternatively, the pellets may be steered so as to spread out during their descent to the liquid surface by, for example, gas jets, by electrostatic deflection, or by a vortexing cone. Dispensing the pellets in single file rather than as a spray may help ensure independent thawing. As with freezing, warming rates within the liquid are determined by pellet size, and so small smaller pellet sizes are preferred.

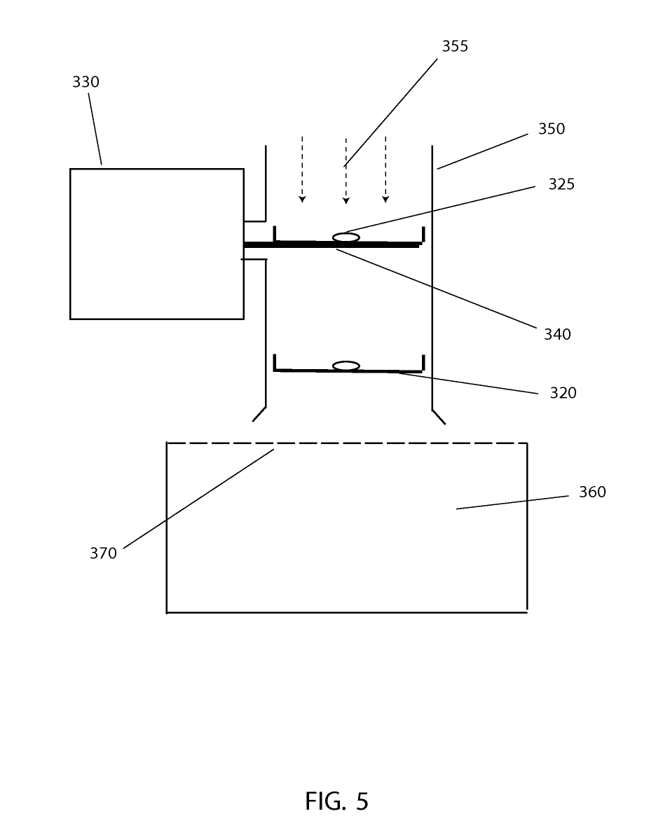

FIG. 5 shows an embodiment of a device for rapid thawing of samples frozen on solid plates or cups 320 as in FIG. 2. Again, the fastest thawing will be achieved by flowing cold gas along the plunge path of the sample, so as to ensure that all thawing occurs once the sample contacts the warming medium. In the embodiment of FIG. 5, frozen samples 325 on metal plates or metal cups 320 are stored in a cold insulated container 330. The cups are individually withdrawn from the chamber using transfer arm 340 and inserted into the tube 350 in which cold gas 355 flows. The cups may then be released and driven downward by gravity and gas pressure, or mechanically driven using vertical translation stage.

When the cups reach the heated metal block 360, they are pushed into contact by the cold flowing gas. They may also be pulled into contact by suction through holes 370 in the block into tight contact with the block, maximizing heat transfer. The cups may have vanes or guides that may match to guides in the tube 350 to produce a smooth motion to the warming surface. The fastest thawing can be ensured by forming the cups from a very thin (e.g., 25 micrometer) sheet of a high thermal conductance metal like copper, to minimize thermal mass and maximize thermal conductance. Even though the sample is only warmed from one side, the superior thermal conduction of the metal can provide much faster warming than total immersion in a liquid, provided that the thickness of the frozen sample on the copper is not too large. “Slamming” cups into a warm block in this way gives a thawed sample that is undiluted and easily retrieved.

The cold flowing gas will retard sample warming once it contacts the heating block to some extent. By insulating the tube 350, the gas flow speed can be reduced while keeping the sample cold until it reaches the heating block. A shutter or vane shielding the sample from the gas flow could be swung into place just above the sample when sample contact with the cooling block is detected.

The present invention has several advantages over prior art systems for cryopreservation. Optimal conditions for maintaining sample viability can be achieved that balance the requirements for rapid cooling and thawing, minimal dehydration and oxygen contamination, and minimal shear forces that may damage cells and other biological samples.

Drop volumes are accurately controlled and the freezing and thawing of each drop is highly consistent. This precision makes interpretation of freeze-thaw experiments much easier and allows more rapid optimization of solution composition and cooling and thawing parameters to maximize sample recovery.

Small drops—with surface area-to-volume ratios above ˜1000 m⁻¹—are used. In prior art systems, small drops are used because it has been expected that they will cool and thaw faster. But the cold gas layers that form above cold surfaces can limit cooling rates for small drops. By removing the cold gas layer to produce a large, abrupt temperature transition at the cooling surface, the present invention actually delivers the much larger cooling rates that smaller drops can in principle provide—from 100 K/s to 100,000 K/s.

For a desired cooling rate, drop size can be thus increased, reducing evaporation and dehydration during its transit to the cooling medium. Drop impact speed with the cooling surface can be reduced, yielding more nicely formed drops and minimizing shear stresses that may damage cells. By flowing warm dry gas across the surface, the dispensing tip can be placed very close to the surface, further reducing evaporation during dispensing and maintaining the integrity of the dispensed solution. The use of warm dry gas also eliminates water vapor condensation and icing, which on thawing would otherwise lead to sample dilution.

Because large cooling rates can be achieved without using very small drops, atomizers and nebulizers that can increase dissolved oxygen content and introduce damaging shear stresses are unnecessary. Faster cooling reduces the need for cryoprotectants that may be hostile to the biological sample.

Although most prior effort has focused on cooling, the warming rates during thawing are almost as critical, as many of the damaging processes that occur during cooling can occur during thawing. The same principles discussed above can be used to thaw frozen samples at the highest possible rates. The key again is to provide a very abrupt temperature transition at the warming surface by flowing cooled gas along the path of the frozen sample, and maximizing thermal conduction to the sample once it contacts the warming medium.

Although the invention has been disclosed in terms of a number of preferred embodiments and variations thereon, it will be understood that numerous additional variations and modifications could be made thereto without departing from the scope of the invention as defined by the following claims.

1. A system for precision freezing cooling and freezing/vitrification of liquids containing biological components, especially those that are sensitive to cooling rate, changes in solute and solvent concentrations and to degradation of the biological components by oxygen, comprising:
a dispenser for converting a liquid sample into a plurality of uniform separated drops of volume between 0.01 nl and 10 ul a surface area-to-volume ratio of ˜1000 m⁻¹ or larger, said dispenser including a dispensing tip for directing said drops onto a cold surface for rapidly cooling said drops; and
means for removing a cold gas layer that forms between said dispensing tip and said cold surface to minimize cooling of each of said drops before they reach said cold surface.

2. The system of claim 1, further including a container in which said dispensing tip and said cold surface are disposed to facilitate control of the atmosphere between the dispensing tip and the cold surface.

3. The system of claim 2, wherein said container contains a dry, oxygen free gas to eliminate condensation on the cold surface, water uptake by the drops, and resulting changes in concentrations within the drops.

4. The system of claim 1, wherein means are provided to cause said drops to land individually and sequentially on different regions of said cold surface.

5. The system of claim 3, where said drops are directed to different regions of the cold surface by one or more of motion of the dispensing tip, by gas jet or electrostatic deflection, and/or by motion of said cold surface.

6. The system in claim 1, wherein said dispenser is a non-gas entrainment type of dispenser selected from the group including a mechanical displacement pump dispenser, a cytometer, a thermal heating based dispenser and a hydrostatic pressure jump based dispenser.

7. The system of claim 1, wherein means are provided for displacing said dispensing tip towards the cold surface during dispensing to increase the drop velocity when it hits the cold surface without increasing shear forces that can be damaging to cells contained within said drops during dispensing.

8. The system of claim 1, wherein said dispensing tip is held between 1 cm and 10 cm from said cold surface to minimize the time during which evaporation can occur from drops dispensing to contact with the cold surface, and to minimize concentration changes in the drop due to evaporation.

9. The system of claim 1, wherein said means for removing said cold gas layer comprises means for blowing a dry oxygen-free gas along said cold surface to eliminate concentration changes due to water vapor condensation, and to produce a large and abrupt temperature change from the initial drop temperature to the temperature of the cold surface, thereby ensuring that nearly all drop cooling from its dispensed temperature occurs in the liquid or on the solid surface at a rate determined by the liquid or solid surface, thereby achieving the shortest possible cooling times for the drops.

10. The system of claim 9, wherein means for blowing pulses said gas stream on and off.

11. The system of claim 9, wherein the magnitude of the temperature change is greater than the difference between one of the melting point of the liquid drop, the homogeneous ice nucleation temperature in the liquid drop or the glass transition temperature and the temperature of the cold surface.

12. The system of claim 1, where said cold surface is formed by a surface of at least partially liquid cryogenic liquid or a hydrocarbon refrigerant.

13. The system of claim 12, wherein said cryogenic liquid is selected from the group comprising liquid nitrogen, liquid propane and liquid ethane.

14. The system of claim 1 wherein said cold surface is formed of a surface of a material selected form the group including dry ice, solid nitrogen, a metal, a metal with an thin inert metal coating, and a metal with a thin inert polymer coating.

15. The system of claim 14, where said cold surface is a surface of a thin-walled metal plate or cup.

16. The system of claim 14, where said plate or cup has a wall that is less than 200 micrometers thick.

17. The system of claim 1, further comprising a container for storing frozen drops at cryogenic temperature and a system for recovering said frozen drops by rapid thawing.

18. The system of claim 17, wherein said system for recovering said frozen drops includes means for collecting said frozen drops from said container and projecting said drops within a cold dry oxygen free gas stream into a warm liquid.

19. The system of claim 18, wherein the drop speed upon reaching the cold liquid surface is 0.1 to 1.0 m/s.

20. The system of claim 18, wherein the temperature of the cold gas stream is below anyone of the melting temperature of the drops, the homogeneous ice nucleation temperature of the drops and the vitrification temperature of the drops.

21. The system of claim 18, wherein said means for projecting said drops in said gas stream pulses said gas stream to keep a surface of said liquid from freezing.

22. The system of claim 18, wherein the warm liquid is a buffer solution.

23. The system of claim 18, wherein the warm liquid is a hydrocarbon based liquid in which the drop constituents are not soluble, so that the thawed drop can be easily separated.

24. A system of claim 1 for recovering cryogenically frozen drops of liquid containing biological components by rapid thawing comprising:
means for collecting frozen drops from a cryogenic container; and
means for projecting said drops within a cold dry oxygen free gas stream into a warm liquid.

25. The system of claim 24, wherein the drop speed upon reaching the cold liquid surface is 0.1 to 1.0 m/s.

26. The system of claim 24, wherein the temperature of the cold gas stream is selected to be below any one of the melting temperature of the drops, the homogeneous ice nucleation temperature of the drops and the vitrification temperature of the drops.

27. The system of claim 24, wherein said means for projecting said gas stream pulses said gas stream to keep a surface of said liquid from freezing.

28. The system of claim 24, wherein the warm liquid is a buffer solution.

29. The system of claim 24, wherein the warm liquid is a hydrocarbon based liquid in which the drop constituents are not soluble, so that the thawed drop can be easily separated.
